(19) United States
(12) Patent Application Publication
(10) Pub. No.: US 2005/0171498 A1
(43) Pub. Date: Aug. 4, 2005

(54) Disposable diaper and method therefor

(76) Inventors: Edward Reiss, Westport, CT (US); Brenda J. Schenk, Westport, CT (US)

Correspondence Address: WEISS & MOY PC, 4204 NORTH BROWN AVENUE, SCOTTSDALE, AZ, 85251, US

(21) Appl. No.: 10/768,376

(22) Filed: Jan. 30, 2004

(51) Int. Cl. A61F 13/15; A61F 13/20

(52) U.S. Cl. 604/385.01

(57) ABSTRACT

A chlorine free disposable diaper having a multi-layered diaper assembly has an exterior layer comprising a non-woven material having a planar, soft, cloth-like surface layer. A core layer is provided and is made of non-chlorine bleached material for holding waste materials within the disposable diaper. A containment layer is coupled to a first side and a second side of the core layer. The containment layer is used for containing the waste material in the core layer and away from skin of a wearer. A distribution layer is coupled to the containment layer. The distribution layer is used for evenly distributing waste material to the core layer. An interior layer is coupled to the distribution layer. The interior layer is made of a non-woven liquid permeable material.

### RELATED APPLICATIONS

The present application is related to U.S. patent application entitled, “DISPOSABLE DIAPER AND METHOD THEREFOR”, having Ser. No. 10/109,091, and a filing date of Mar. 27, 2002 and U.S. Pat. No. 5,743,895 entitled “DISPOSABLE DIAPER AND METHOD THEREFOR”, issued on Apr. 28, 1998 in the name of the same inventors and incorporated by reference into the present application.

### BACKGROUND OF THE INVENTION

1. Field of the Invention

This invention relates to a disposable diaper and, more specifically, to a disposable diaper and method therefor that is totally chlorine free.

2. Description of the Prior Art

Disposable diapers are well known and widely used. Over the years, disposable diapers have become the dominant diaper, largely replacing the old form of reusable diaper. Typical disposable diapers generally use an absorbent interior area for absorbing a wearer's urine, bounded by a thin plastic exterior layer. The interior portion of the typical disposable diaper presently on the market is generally comprised of a bottom layer of non-woven material that is in direct contact with the area of the child's skin covered by the diaper, a backing layer of tissue paper that contacts the bottom portion of the layer of non-woven material, a wadding batt layer of wood pulp that is located between the thin plastic exterior layer and the layer of tissue paper, and, in order to increase the diaper's fluid absorbency, diaper manufacturers add a superabsorbent polymer or other chemical additive to the wadding batt layer. Without the superabsorbent polymer, wood pulp generally absorbs in the range of 12 to 15 times its weight. With the superabsorbent polymer, the wadding batt layer is able to absorb in the range of 30 to 55 times its weight.

One problem with current disposable diapers is that the wood pulp used is bleached with chlorine. This bleaching process produces low levels of dioxins or furons as by-products. While the government may allow certain low levels of dioxins, dioxins are toxic and carcinogenic. Some companies use ECF elemental chlorine free bleached wood pulp. ECF elemental chlorine free bleached wood pulp still has small trace amounts of chlorine. Most governmental agencies allow these small trace amounts and allow companies to claim that there products are chlorine free. However, as stated above, products that contain ECF elemental chlorine free bleached wood pulp still has small trace amounts of chlorine.

As stated above, most disposable diapers use a backing layer of tissue paper that contacts the bottom portion of the layer of non-woven material. This tissue paper is generally a bleached tissue layer. The tissue paper is bleached using a chlorine bleach process. As stated above, the chlorine bleaching processes produces low levels of dioxins or furons as by-products. Furthermore, the bleached tissue paper may also contain small trace amounts of chlorine.

Therefore, a need existed to provide an improved disposable diaper and method that overcome the problems associated with prior art disposable diapers. The improved disposable diaper and method must be totally chlorine free. The improved disposable diaper and method must use materials that do not use a chlorine bleach during the manufacturing process.

### SUMMARY OF THE INVENTION

In accordance with one embodiment of the present invention, it is an object of the present invention to provide an improved disposable diaper and method.

It is another object of the present invention to provide an improved disposable diaper and method that overcomes the above problems associated with prior art disposable diapers.

It is still another object of the present invention to provide an improved disposable diaper and method that is totally chlorine free.

It is still another object of the present invention to provide an improved disposable diaper and method that uses materials that are not chlorine bleached during the manufacturing process.

### BRIEF DESCRIPTION OF THE EMBODIMENTS

In accordance with one embodiment of the present invention a chlorine free disposable diaper having a multi-layered diaper assembly is disclosed. The disposable diaper has an exterior layer comprising a non-woven material having a planar, soft, cloth-like surface layer. A core layer made of non-chlorine bleached material is provided for holding waste materials within the disposable diaper. A containment layer is coupled to a first side and a second side of the core layer for containing the waste material in the core layer and away from skin of a wearer. A distribution layer is coupled to the containment layer for evenly distributing waste material to the core layer. An interior layer is coupled to the distribution layer comprising a non-woven liquid permeable material.

In accordance with another embodiment of the present invention, a method for providing a chlorine free disposable diaper having a multi-layered diaper assembly is disclosed. The method comprising: forming an exterior layer comprising a non-woven material having a planar, soft, cloth-like surface layer; coupling a leakage protection layer to the exterior layer for preventing waste material from leaking out of the disposable diaper; forming a core layer made of non-chlorine bleached wood pulp for holding the waste materials within the disposable diaper; coupling a containment layer to a first side and a second side of the core layer for containing the waste material in the core layer and away from skin of a wearer; coupling a distribution layer to the containment layer for evenly distributing waste material to the core layer; and coupling an interior layer to the distribution layer comprising a non-woven liquid permeable material.

The foregoing and other objects, features, and advantages of the invention will be apparent from the following, more particular, description of the preferred embodiments of the invention, as illustrated in the accompanying drawing.

### BRIEF DESCRIPTION OF THE DRAWINGS

The novel features believed characteristic of the invention are set forth in the appended claims. The invention itself, as well as a preferred mode of use, and advantages thereof, will best be understood by reference to the following detailed description of illustrated embodiments when read in conjunction with the accompanying drawings.

FIG. 1 is an elevated front side view of one embodiment of the disposable diaper of the present invention.

FIG. 2 is an elevated rear side or backside view of one embodiment the disposable diaper of the present invention.

FIG. 3 is a cross-sectional view of the disposable diaper of FIG. 1 taken along line 3-3 of FIG. 1.

### DETAILED DESCRIPTION OF THE PREFERRED EMBODIMENT

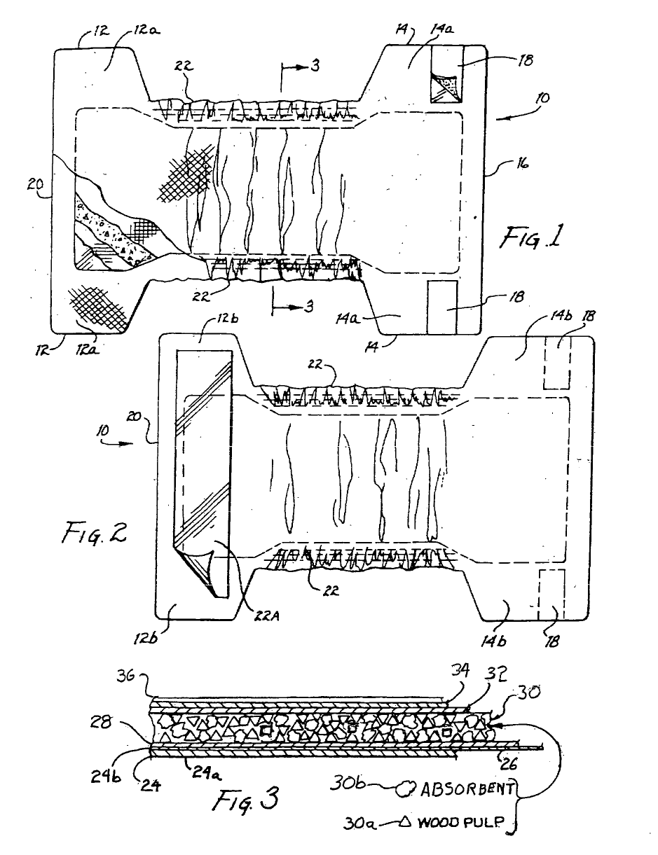

Referring to the embodiment of FIGS. 1-3, reference number 10 refers generally to the disposable diaper of this invention. It should be noted that the following description is one embodiment of the present invention.

Referring to FIG. 1, a front or interior view of the disposable diaper 10 is shown, in the open position. The disposable diaper 10, like other disposable diapers, comprises front flaps 12 and back flaps 14. Reference numbers 12a and 14a refer to the side portions of the front flaps 12 and the back flaps 14, respectively, that are visible in the front view of the disposable diaper 10 depicted in FIG. 1. Adhered to the back flaps 14a, and oriented substantially parallel to an upper edge 16 of the diaper 10, are preferably two tape assemblies 18. Each tape assembly 18 comprises a base portion that is attached directly to the surface of the diaper 10, and a partially separable adhesive portion that may be peeled from the base portion for adhesion attachment to the reverse side of front flaps 12 (see both FIGS. 1 and 2) when the diaper 10 is being placed on a baby or child. On both sides of the diaper 10, oriented in a substantially perpendicular direction to the upper edge 16 and the lower edge 20, are two elastic portions 22. The elastic portions 22 snugly grip the legs of the child during the wearing of the diaper 10 to, among other things, minimize the leaking of fluid out of the diaper 10.

Referring to FIG. 2, a rear or backside exterior view of the disposable diaper 10 is shown, in the open position. Reference numbers 12b and 14b refer to the sides of the front flaps 12 and the back flaps 14, respectively, that are visible in the rear or backside view of the disposable diaper 10. Adhered to the back flaps 14b, and oriented substantially parallel to the upper edge 16 of the diaper 10, are portions of the two tape assemblies 18. These are continuations of the portions of the tape assemblies 18 which are located on the back flaps 14a, as shown in FIG. 1. Located between tabs 12b is a preferably rectangular plastic strip 22A, which strip 22A receives the adhesive portion of the tape assemblies 18 when the diaper is being placed on a baby or child.

Referring to FIG. 3, a cross-sectional view of the disposable diaper 10 is shown, showing the layers of material comprising the diaper 10. The disposable diaper 10 general has a plurality of different layers. The first layer 24 has an outer surface 24a and an inner surface 24b. The outer surface 24a is generally exposed. Since the outer surface 24a is exposed, the first layer 24 is used primarily for aesthetics. In accordance with one embodiment of the present invention, the first layer 24 is made of a spunbond hydrophobic non-woven polypropylene material having a soft, cloth-like appearance.

A second layer 26 is provided and also has an inner and outer surface. The outer surface of the second layer 26 is coupled to the inner surface 24b of the first layer 24. The second layer 24 is used to contain any fluids or solids within the disposable diaper 10. Thus, the second layer 24 helps to prevent leakage. The second layer 24 is generally made from a polyethylene material.

A third layer 28 is coupled to the inner surface of the second layer 24. The third layer has an outer surface which is directly coupled to the inner surface of the second layer 24. The third layer 28 is also used to contain any fluids and material within the disposable diaper 10. The third layer 28 further provides an area to keep fluids and other material inside the disposable diaper 10 away from the skin of the wearer of the disposable diaper 10. The third layer 28 is generally made of a spunbond hydrophilic non-woven material. All disposable diapers currently use a bleached tissue. However, the disposable diaper 10 will use a non bleached spunbond hydrophilic non-woven material as the third layer 28.

Coupled to an inner surface of the third layer 28 is a fourth layer 30. The fourth layer 30 is a wadding batt layer consisting of a wood pulp mixture 30a. All present disposable diapers bleach their wood pulp with chlorine. This bleaching process low levels of dioxins. Even though low levels of dioxins are accepted by the government, many people would prefer to have a disposable diaper 10 made from non-chlorine bleached materials. Thus, in accordance with one embodiment of the present invention, a non-chlorine bleached wood pulp 30a is used. A bleached untreated softwood fluff pulp which is biodegradable and totally chlorine free may also be used. Additionally, wood pulp 30a used in the present invention may be bleached using other processes other than chlorine. In accordance with one embodiment of the present invention, the wood pulp 30a is bleached with a hydrogen peroxide mixture. The use of hydrogen peroxide should not be seen as to limit the scope of the present invention.

The fourth layer 30 may further comprise a superabsorbent mixture 30b. The superabsorbent mixture 30b will allow the wadding batt layer to absorb in the range of 30 to 55 times its weight. However, instead of a superabsorbent polymer such as polyacrylates, one that is more friendly to the skin would be used. The superabsorbent mixture 30b may be a natural or an artificial superabsorbent mixture 30b. The superabsorbent mixture 30b will still allow the wadding batt layer to absorb in the range of 30 to 55 times it weight, but will not have the side effects of irritating the wearer's skin during “rewet”.

A fifth layer 32 is coupled to the fourth layer 30. The fifth layer 32 is similar to that of the third layer 28. The fifth layer 32 and the third layer 28 are used in combination to contain any fluids and material within the fourth layer 30. The fifth layer 32 further provides an area to keep fluids and other material inside the disposable diaper 10 away from the skin of the wearer of the disposable diaper 10. The fifth layer 32 is generally made of a spunbond hydrophilic non-woven material. All disposable diapers currently use a bleached tissue. However, the disposable diaper 10 will use a non bleached spunbond hydrophilic non-woven material as the fifth layer 32.

A sixth layer 34 is coupled to one side of the fifth layer 32. The sixth layer 34 is an acquisition distribution layer. The sixth layer 34 is used to distribute any fluids evenly through the fourth layer 30 and to keep the surface of the sixth layer 34 dry. In accordance with one embodiment of the present invention, the sixth layer 34 will use a non bleached non-woven material.

A seventh layer 36 is coupled to the sixth layer 34. The seventh layer 36 is a top sheet which is the closest layer to the skin of the wearer. The seventh layer 36 is generally made of a spunbond polypropylene hydrophilic non-woven material. The seventh layer 36 is used to carry the fourth layer 30 through machine and provides a soft layer against the user's skin. The seventh layer 36 will further be a non-woven liquid permeable material.

By using spunbond hydrophilic non-woven material and a TCF chlorine free woodpulp, a disposable diaper 10 is produced which is 100% chlorine free.

While the invention has been particularly shown and described with reference to preferred embodiments thereof, it will be understood by those skilled in the art that the foregoing and other changes in form and details may be made therein without departing from the spirit and scope of the invention.

1. A chlorine free disposable diaper having a multi-layered diaper assembly comprising:
an exterior layer comprising a non-woven material having a planar, soft, cloth-like surface layer;
a core layer made of non-chlorine bleached material for holding waste materials within the disposable diaper;
a containment layer coupled to a first side and a second side of the core layer for containing the waste material in the core layer and away from skin of a wearer;
a distribution layer coupled to the containment layer for evenly distributing waste material to the core layer; and
an interior layer coupled to the distribution layer comprising a non-woven liquid permeable material.

2. A chlorine free disposable diaper having a multi-layered diaper assembly in accordance with claim 1 further comprising a leakage protection layer coupled to the exterior layer for preventing waste material from leaking out of the disposable diaper.

3. A chlorine free disposable diaper having a multi-layered diaper assembly in accordance with claim 1 wherein the core layer comprises a non-chlorine bleached wood pulp material.

4. A chlorine free disposable diaper having a multi-layered diaper assembly in accordance with claim 3 wherein the core layer further comprises a superabsorbent mixture.

5. A chlorine free disposable diaper having a multi-layered diaper assembly in accordance with claim 1 wherein the exterior layer comprises a spunbond hydrophobic non-woven polypropylene material.

6. A chlorine free disposable diaper having a multi-layered diaper assembly in accordance with claim 1 wherein the containment layer comprises a spunbond hydrophilic non-woven material.

7. A chlorine free disposable diaper having a multi-layered diaper assembly in accordance with claim 1 wherein the distribution layer comprises a non bleached non-woven material.

8. A chlorine free disposable diaper having a multi-layered diaper assembly in accordance with claim 1 wherein the interior layer comprises a spunbond hydrophobic non-woven polypropylene material.

9. A chlorine free disposable diaper having a multi-layered diaper assembly comprising:
an exterior layer comprising a non-woven material having a planar, soft, cloth-like surface layer;
a leakage protection layer coupled to the exterior layer for preventing waste material from leaking out of the disposable diaper;
a core layer made of non-chlorine bleached wood pulp for holding the waste materials within the disposable diaper;
a containment layer coupled to a first side and a second side of the core layer for containing the waste material in the core layer and away from skin of a wearer;
a distribution layer coupled to the containment layer for evenly distributing waste material to the core layer; and
an interior layer coupled to the distribution layer comprising a non-woven liquid permeable material.

10. A chlorine free disposable diaper having a multi-layered diaper assembly in accordance with claim 9 wherein the core layer further comprises a superabsorbent mixture.

11. A chlorine free disposable diaper having a multi-layered diaper assembly in accordance with claim 9 wherein the exterior layer comprises a spunbond hydrophobic non-woven polypropylene material.

12. A chlorine free disposable diaper having a multi-layered diaper assembly in accordance with claim 9 wherein the containment layer comprises a spunbond hydrophilic non-woven material.

13. A chlorine free disposable diaper having a multi-layered diaper assembly in accordance with claim 9 wherein the distribution layer comprises a non bleached non-woven material.

14. A chlorine free disposable diaper having a multi-layered diaper assembly in accordance with claim 9 wherein the interior layer comprises a spunbond hydrophobic non-woven polypropylene material.

15. A method of providing a chlorine free disposable diaper having a multi-layered diaper assembly comprising:
forming an exterior layer comprising a non-woven material having a planar, soft, cloth-like surface layer;
coupling a leakage protection layer to the exterior layer for preventing waste material from leaking out of the disposable diaper;
forming a core layer made of non-chlorine bleached wood pulp for holding the waste materials within the disposable diaper;
coupling a containment layer to a first side and a second side of the core layer for containing the waste material in the core layer and away from skin of a wearer;
coupling a distribution layer to the containment layer for evenly distributing waste material to the core layer; and
coupling an interior layer to the distribution layer comprising a non-woven liquid permeable material.

16. The method of claim 15 wherein the core layer further comprises a superabsorbent mixture.

17. The method of claim 15 wherein the exterior layer comprises a spunbond hydrophobic non-woven polypropylene material.

18. The method of claim 15 wherein the containment layer comprises a spunbond hydrophilic non-woven material.

19. The method of claim 15 wherein the distribution layer comprises a non bleached non-woven material.

20. The method of claim 15 wherein the interior layer comprises a spunbond hydrophobic non-woven polypropylene material.
